(19) United States
(12) Patent Application Publication
(10) Pub. No.: US 2009/0099783 A1
(43) Pub. Date: Apr. 16, 2009

(54) Stage Specific Prognostic In Vivo Markers of Brain Aging and Dementia

(76) Inventor: Barry Reisberg, New York, NY (US)

Correspondence Address: FAY KAPLUN & MARCIN, LLP, 150 BROADWAY, SUITE 702, NEW YORK, NY, 10038, US

(21) Appl. No.: 12/134,768

(22) Filed: Jun. 6, 2008

Related U.S. Application Data
(60) Provisional application No. 60/942,960, filed on Jun. 8, 2007.

(51) Int. Cl. G06F 19/00; A61B 10/00

(52) U.S. Cl. 702/19; 424/9.2

(57) ABSTRACT

A method for the identification of treatments and preventative agents for brain aging, subjective cognitive impairment (SCI), mild cognitive impairment (MCI), Alzheimer's disease (AD) and other degenerative dementias, the method including (a) the identification of the diagnosis and stage of the subject, (b) the identification of the duration of the stage of the condition and/or disorder, (c) the identification of prognostic markers based upon a formula incorporating the duration of the condition and/or stage, (d) the prospective separation of prognostic subgroups based upon outcome wherein the outcome is defined in these conditions as progression to a subsequent stage or stages, (e) the employment of a putative prognostic marker for an appropriate period of time, based upon the formula incorporating the duration of the condition and/or stage, (f) the application of in vivo, methodology specific techniques, in conjunction with stage specific prognostic subgroups, for the appropriate time period, for the identification, prospectively, of useful markers, (g) the employment of these markers to identify useful therapeutic agents for prevention and treatment.

### PRIORITY CLAIM

This application claims the priority to the U.S. Provisional Application Ser. No. 60/942,960, entitled “Stage Specific Prognosis in Vivo Markers,” filed Jun. 8, 2007. The specification of the above-identified application is incorporated herewith by reference.

### FIELD OF INVENTION

The present invention relates to a system and method for the identification of in vivo markers for the treatment of Alzheimer's disease and other cognitive dysfunctions.

### BACKGROUND INFORMATION

The clinical assessment of the cognitive and functional capacity of patients for the diagnosis of degenerative disorders has historically relied upon the results of neurocognitive memory tests and clinical interviews. Examples of such tests include global clinical staging measures such as the Global Deterioration Scale (“GDS”) (Reisberg et al., 1982), the Blessed Dementia Scale and Information-Memory-Concentration Test, the Mini Mental Status Examination (MMSE) (Folstein M F et al., 1975), the Alzheimer's Disease Assessment Scale (“ADAS”), and the Functional Assessment Staging Scale (“FAST”) (Reisberg et al., 1984, Reisberg, 1988).

The success of available methods for the staging of cognitive disorders and for predicting the rate of future decline has been limited by the lack of sensitive in vivo markers in current imaging and other modalities.

### SUMMARY OF THE INVENTION

The present invention is directed to a system and method for the identification of treatments and preventative agents for brain aging with no cognitive impairment (NCI), subjective cognitive impairment (SCI) (synonymously, this condition is sometimes referred to as one of subjective cognitive complaints [other terms which are less precise and sometimes used to describe similar conditions to SCI include age-associated memory impairment (AAMI) and age-associated cognitive decline]), mild cognitive impairment (MCI), Alzheimer's disease (AD) and other degenerative dementias. The present invention is further directed to a system and method for the identification of a GDS and FAST stage of a subject and for employing a putative prognostic marker for an appropriate period of time, based upon a formula incorporating the duration of the condition and/or stage. Furthermore, the present invention is directed to the application of in vivo, methodology specific techniques, in conjunction with stage specific prognostic subgroups, for the appropriate time period, for the identification of useful markers, which may identify useful therapeutic agents for prevention and treatment of degenerative dementia.

### BRIEF DESCRIPTION OF THE DRAWINGS

FIG. 1 shows a chart relating the progression of the Brief Cognitive Rating Scale (BCRS) stages to the GDS stages of brain aging and AD;

FIG. 2A shows the relationships between hippocampal volumes and FAST substages,

FIG. 2B shows the precise neuropathologic relationships between hippocampal regional volumes, hippocampal regional neuronal cell loss, and hippocampal regional neurofibrillary changes and FAST substages;

FIG. 3 shows the percentages of remaining neurons with neurofibrilliary changes in the hippocampal brain regions with the progression of the FAST substages, based upon postmortem neuropathologic studies;

FIG. 4 shows the relationship between changes in assessment measures and time to follow up in persons with AD;

FIG. 5 shows the progressive slowing of EEG frequency bands of subjects with progressive GDS stages;

FIG. 6 is a chart showing FAST and time course of functional loss in normal brain aging with NCI, subjective cognitive impairment (SCI), mild cognitive impairment (MCI), and Alzheimer's Disease.

FIG. 7 shows the relationship between predicted and actual time to follow up in survivors of AD via GDS;

FIG. 8 shows the relationship between predicted and actual time to follow up in survivors of AD via FAST;

FIG. 9 shows the imaging method used in the present invention to predict change along the brain aging and degenerative dementia continuum; and

FIG. 10 shows an exemplary method according to the present invention.

### DETAILED DESCRIPTION

The present invention may be further understood with reference to the following description and the appended drawings. An exemplary embodiment of the present invention is directed to a system and method for identifying stage specific treatments and preventative agents for brain aging with no cognitive impairment (“NCI”), Subjective Cognitive Impairment (“SCI”), Mild Cognitive Impairment (“MCI”), Alzheimer's Disease (“AD”) and other degenerative dementias.

### Stages of Brain Aging and Alzheimer's Disease

Conventional GDS techniques are used to summarize whether an individual has cognitive impairments consistent with a degenerative dementia such as AD. The GDS system categorizes exhibited symptoms into one of seven global stages. The seven stages range from stage 1, wherein a patient exhibits no subjective memory deficit to stage 7, wherein a patient may have lost all basic psychomotor skills and verbal abilities and will exhibit incontinence. Stages 2-6 are indicative of the range of aging and dementia lying therebetween. Despite the wide usage of the GDS system, there has been a continuing need to describe the prognostic import of these seven stages.

Boundaries between normal aging and Alzheimer's Disease have been clarified via disclosure of the detailed mental status and psychometric concomitants of the GDS stages which indicate clearly that the terminology “dementia” was appropriate to GDS stages 4 and above (Reisberg et al., Stage-specific behavioral, cognitive, and in vivo changes in community residing subjects with age-associated memory impairment and primary degenerative dementia of the Alzheimer type”, Drug Development Research, 1988, 15:101-114.). Alternate works have further verified these boundaries based upon a five year longitudinal study (Reisberg et al., “Mortality and temporal course of probable Alzheimer's disease: A five-year prospective study.”, International Psychogeriatrics, 1996, 8:291-311).

Simultaneously, with the development of the GDS stages, other behavioral and clinical markers of the progression of brain aging and AD have also been identified. These stage specific, optimally concordant measures of progressive change became the GDS staging system (Reisberg et al. “Clinical stages of normal aging and Alzheimer's disease: The GDS staging system.”, Neuroscience Research Communications, 1993, 13 (Suppl. 1): 551-554). The GDS staging system consists of the Brief Cognitive Rating Scale (“BCRS”) (Reisberg et al. “The brief cognitive rating scale (BCRS): Findings in primary degenerative dementia (PDD)”, Psychopharmacology Bulletin, 1983, 19:47-50; Reisberg et al., The Brief Cognitive Rating Scale (BCRS). Psychopharmacology Bulletin, 1988, 24:629-636) and the Functional Assessment Staging Scale (“FAST”) (Reisberg et al. “Functional staging of dementia of the Alzheimer's type.” Annals of the New York Academy of Sciences, 1984, 435:481-483; Reisberg, “Functional assessment staging (FAST).” Psychopharmacology Bulletin, 1988, 24:653-659).

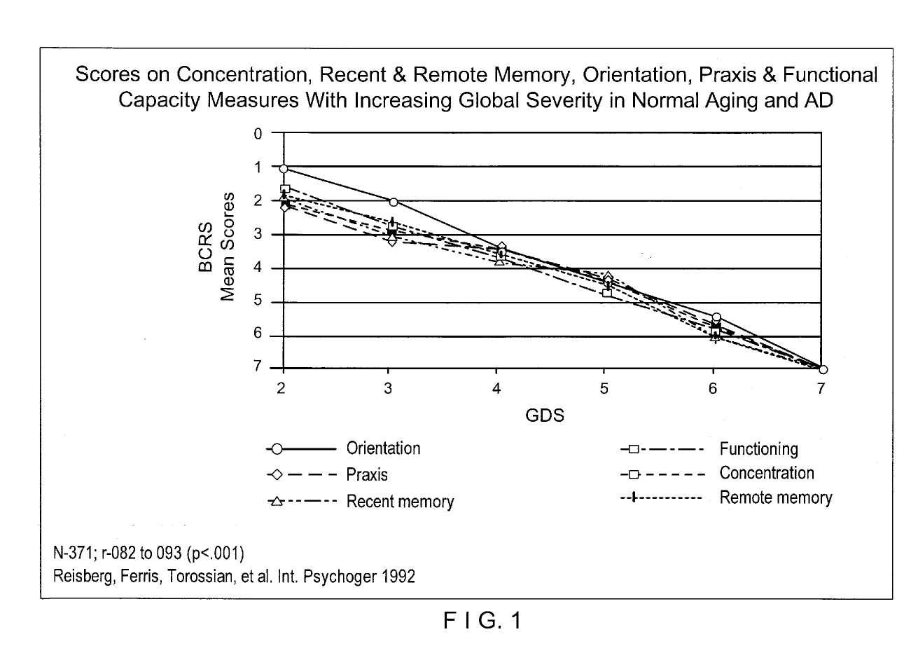

The BCRS method is comprised of measures of concentration and calculation ability, recent memory retention, remote memory retention, orientation, praxis, and functional capacity in a patient, as those skilled in the art will understand. The FAST method is directed to dividing the progression of a degenerative dementia into 16 successive stages and substages under the 7 major headings of functional abilities and losses, as defined by the GDS. As shown in FIG. 1, studies have demonstrated the optimal concordance of the BCRS and FAST techniques with the progression of cognitive and cognition related functional change in aging and AD. Specifically, FIG. 1 demonstrates the relationship between a deteriorating GDS scale and a mean BCRS score in a sample of 371 subjects, wherein the subjects were free of medical psychiatric, neurologic or neuroradiologic conditions, or any other recognizable condition that might interfere with cognition apart from SCI, MCI, or probable AD. Using various procedures, including neuropathologic studies, as those skilled in the art will understand, continuing changes in elements of the GDS staging system with the progression of AD are apparent. Although the present invention is described in conjunction with the GDS staging system, including the GDS, BCRS and the FAST, those skilled in the art will understand that the same results may be obtained with any properly constructed and designed staging method.

Linear Changes of the GDS Staging System with the Progression of AD

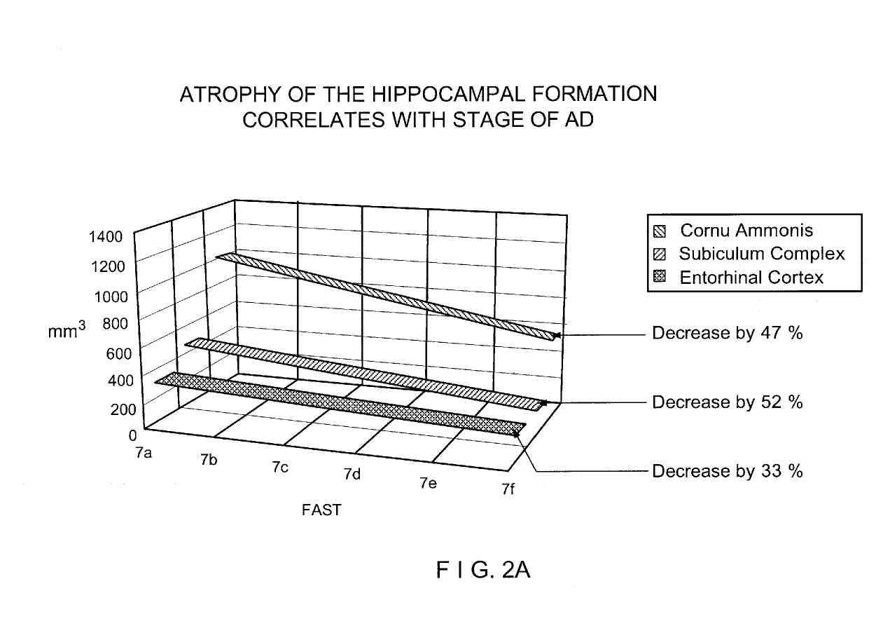

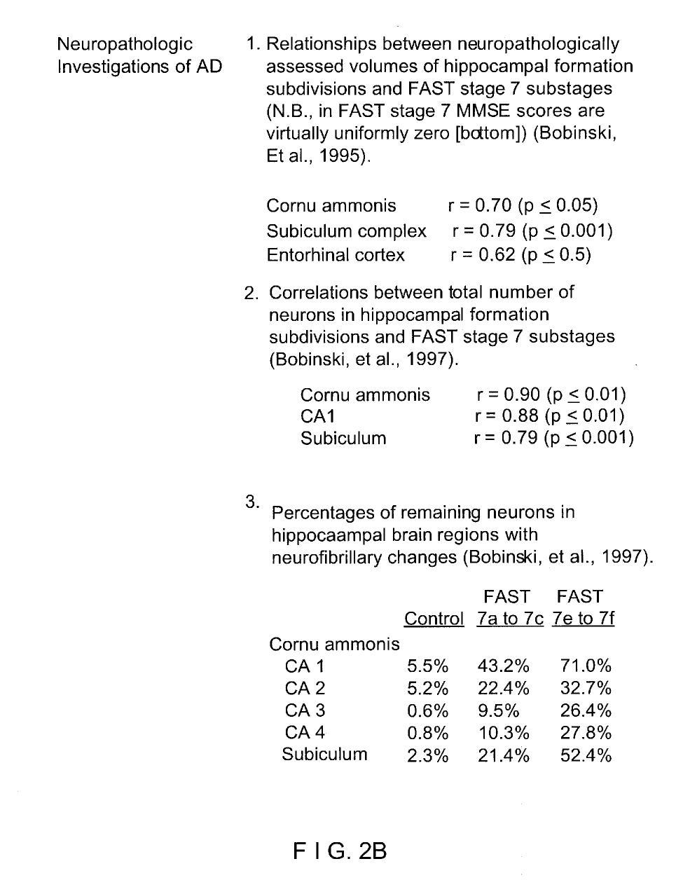

The linear aspects of the GDS staging system are further illustrated in FIG. 2A, which shows the results of published neuropathologic postmortem studies of specific hippocampal brain regions in patients with severe AD (GDS and FAST Stages 7). Specifically, FIG. 2A shows the continuing linear changes in hippocampal brain regions with the progression of the stages and substages of AD. For example, in terms of hippocampal volume changes, as illustrated in FIG. 2B item 1, we observed a correlation between the volume of the subiculum complex and the FAST substages of r=0.79 (p≦0.001), as those skilled in the art will understand. Robust changes in the volume of other hippocampal brain regions with the progression of the FAST substages were also observed. As shown in FIG. 2B item 1, the cornu Ammonis volume showed a correlation with the FAST substage progression of r=0.70 (p≦0.05) and the entorhinal cortex volume changes correlated with the FAST substage progression at r=0.62 (p≦0.05) (Bobinski et al. “Atrophy of hippocampal formation subdivisions correlates with stage and duration of Alzheimer disease.” Dementia, 1995, 6:205-210). The correlation between the volumes of hippocampal brain regions and FAST substages therefore provide strong support for the linear nature of the relationship between the FAST staging procedure and progressive hippocampal regional volumetric changes.

Even stronger linear correlations were noted between progressive changes in neuronal cell numbers in the various hippocampal brain subregions and the progression of AD as assessed with the FAST staging procedure (Bobinski et al. “Relationships between regional neuronal loss and neurofibrillary changes in the hippocampal formation and duration and severity of Alzheimer disease.” Journal of Neuropathology and Experimental Neurology, 1997, 56:414-420). For example, the magnitude of neuronal loss (i.e., cell counts in the specific hippocampal brain regions), correlates with FAST substages up to a magnitude of r=0.90 in the cornu ammonis (p≦0.01) (FIG. 2B item 2). Similarly, as shown in FIG. 2B item 2, robust correlations with neuronal cell loss in hippocampal brain regions were found in the CA1 region of the hippocampus and in the subiculum, as those skilled in the art will understand.

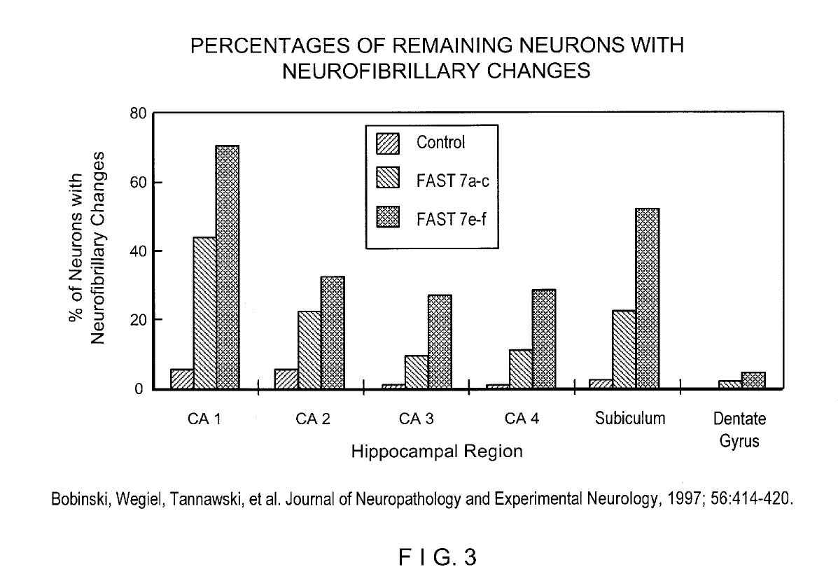

Importantly, neurofibrillary changes have also shown linear relationships within hippocampal brain regions to the progression of Alzheimer's disease assessed in GDS and FAST stage 7 (Bobinski et al. “Relationships between regional neuronal loss and neurofibrillary changes in the hippocampal formation and duration and severity of Alzheimer disease.” Journal of Neuropathology and Experimental Neurology, 1997, 56:414-420). FIG. 3 demonstrates the percentages of remaining neurons with neurofibrillary changes in six hippocampal regions of patients with Alzheimer's disease. As shown in FIG. 2B item 3 and FIG. 3, patients in the early part of FAST stage 7 (FAST stages 7a to 7c) had a far greater percentage of remaining neurons with neurofibrillary involvement than control subjects. Further, these percentages increased dramatically in the various hippocampal brain regions in the latter part of FAST stage 7 (FAST stages 7e to 7f).

Hence fundamental neuropathologic features of AD including changes in hippocampal regional volumes and hippocampal regional neuronal numbers, as well as progressive neurofibrillary pathology in hippocampal brain regions, all proceed linearly with the FAST staging of AD.

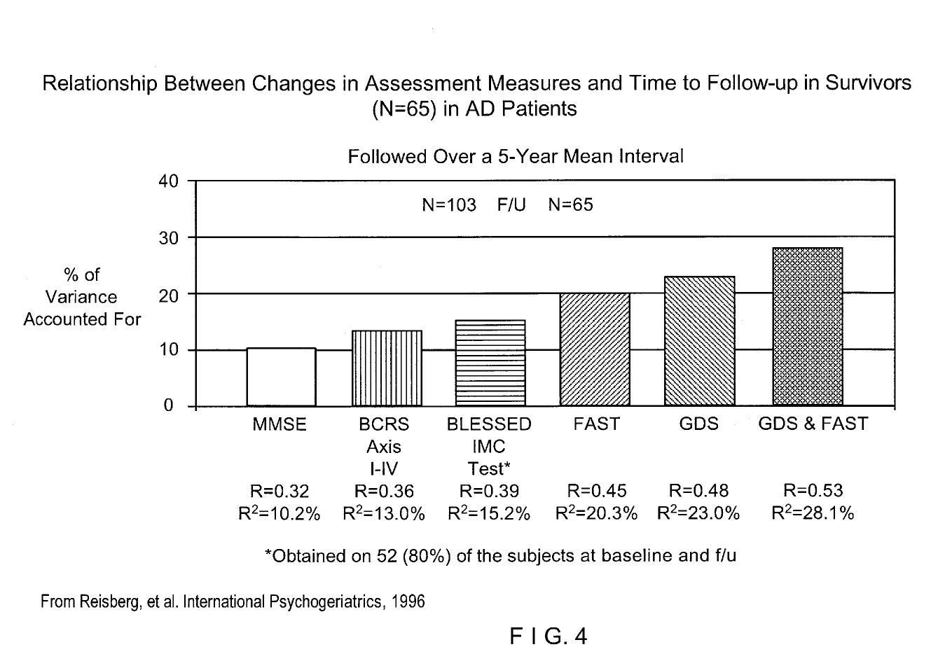

Another important aspect of the linear changes which occur with the progression of the GDS and FAST and the progression of AD, can be seen with temporal change. In the five year prospective longitudinal study noted above (Reisberg et al. “Mortality and temporal course of probable Alzheimer's disease: A five-year prospective study.” International Psychogeriatrics, 1996, 8:291-311), the GDS and FAST together explained approximately 3 times the temporal variance in AD progression as the widely used MMSE (Folstein M F et al. (1975) “Mini-mental state. A practical method for grading the cognitive state of patients for the clinician. Journal of Psychiatric Research, 12:189-198). Superiority to another widely used dementia assessment, the Blessed et al., Information Memory and Concentration Test (Blessed G, Tomlinson B E, Roth M. (1968) “The association between quantitative measures of dementia and of senile change in the cerebral grey matter of elderly subjects.” British Journal of Psychiatry, 114:797-811) was also noted, as is shown with respect to FIG. 4. FIG. 4 shows the relationship between changes in the assessment measures and time to follow-up in AD survivors over a five-year mean interval.

There are several reasons for the superiority of the GDS and FAST staging to the MMSE and the Blessed et al. test in charting the course of AD as shown by the stronger temporal relationship to progression in this progressive disease. These include: (1) the greater range of the GDS and FAST in terms of both ceiling and floor effects; (2) the linear nature of the change in the GDS and FAST with respect to the disease progression; and (3) the superior construction and design of the GDS and FAST in terms of charting the entire course of AD pathology. For example, in the 5-year study of the temporal course of AD described above, subjects at baseline had a mean MMSE score of 15.4±5.6. Of the 103 AD subjects studied at baseline, 92% of subjects were located and followed 5 year later. Of these subjects, 30 were deceased. Of the remainder of subjects who survived, slightly more than half (51%), had bottom (zero) MMSE scores. Similarly, Blessed et al., test scores of 0 (bottom scores) were noted for 50% of the subjects who were assessed at the five year follow-up. In contrast, only 4 of the 65 subjects with AD followed (6.2%) had reached the final FAST substage at the time of the follow-up assessment (Reisberg, et al., “Mortality and temporal course of probable Alzheimer's disease: A five-year prospective study.”, International Psychogeriatrics, 1996, 8:291-311).

In terms of general measurement construction and sensitivity, a pivotal, multi-center study of the efficacy of the medication memantine in the treatment of patients with moderate to severe AD provided a direct comparison of the sensitivity to pharmacologic intervention of the MMSE in comparison with the FAST (Reisberg et al. “Memantine in moderate-to-severe Alzheimer' disease.” New England Journal of Medicine, 2003, 348:1333-1341). At baseline, all subjects in this 32 site, placebo-controlled, multicenter trial had MMSE scores from 3 to 14 and a stage 6a or greater on the FAST. At the conclusion of this 28 week, double blind, parallel group study, in which patients were randomly assigned to treatment with memantine or placebo, the MMSE did not show a significant effect of the medication treatment. In contrast, the FAST improved significantly with the pharmacologic intervention. Using the Last Observation Carried Forward analysis, the FAST showed a significant effect in the memantine treated patients in comparison with the placebo treated patients with a p value of 0.02. Using the Observed Cases analysis, the FAST showed a significant effect of the pharmacologic intervention with a p value of 0.007. Hence, the FAST is a comparatively sensitive and robust marker of the magnitude of symptomatology in AD.

In addition to showing linear changes in AD in terms of neuropathologic volumetric, neuronal loss, and neurofibrillary changes, as well as temporal changes with the progression of AD, there is a need to describe the nature of the earlier GDS and FAST stages. The terminology “mild cognitive decline” was originally suggested for GDS and FAST stages 3 (Reisberg et al., The global deterioration scale for assessment of primary degenerative dementia. American Journal of Psychiatry, 1982, 139:1136-1139). Subsequently, GDS stage 3 was also referred to as “mild cognitive impairment” (Reisberg et al., Stage-specific behavioral, cognitive, and in vivo changes in community residing subjects with age-associated memory impairment and primary degenerative dementia of the Alzheimer type. Drug Development Research, 1988, 15:101-114.) A continuum of change has been demonstrated in patients with MCI in comparison with GDS stage 2 (SCI) patients. A similar change has been demonstrated for MCI subjects in comparison with GDS stage 4 (mild AD) subjects. These differences between GDS stage 3 and the earlier GDS stage 2 as well as the later, GDS stage 4 have been demonstrated in terms of a variety of parameters, including equilibrium and coordination measures, changes in daily activities, mental status, subjective and spousal assessment of cognitive problems, emotional problems and functional changes, etc. (Reisberg, et al., Mild cognitive impairment (MCI): A historical prospective. International Psychogeriatrics, 2008, 20:18-31.)

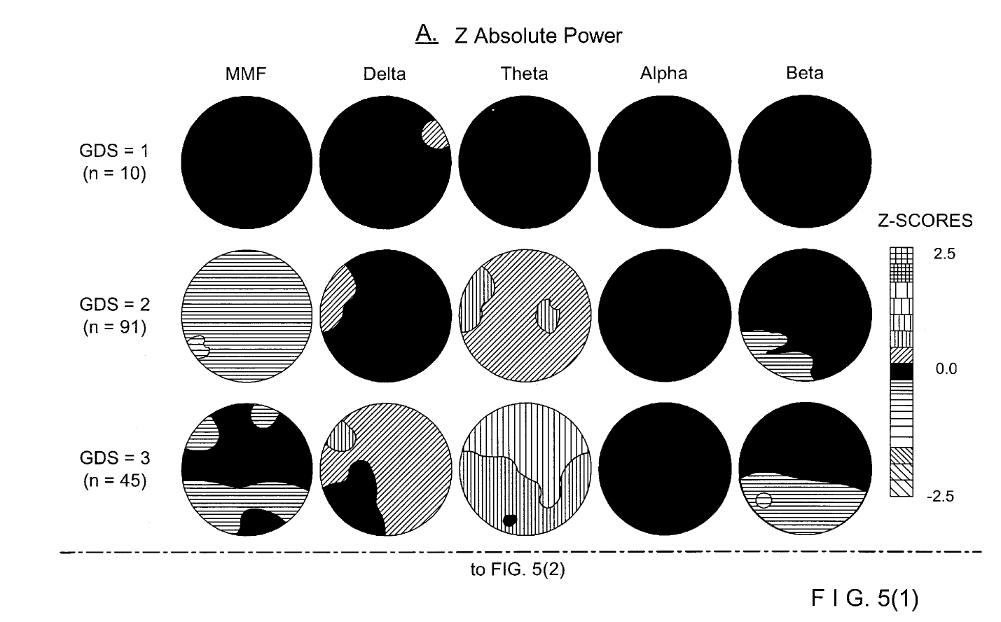

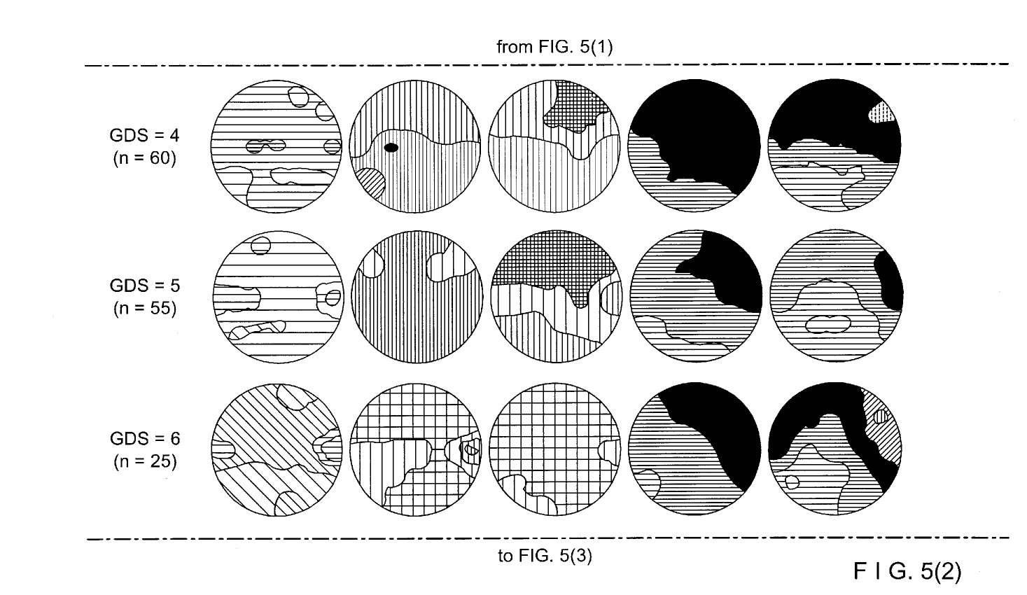

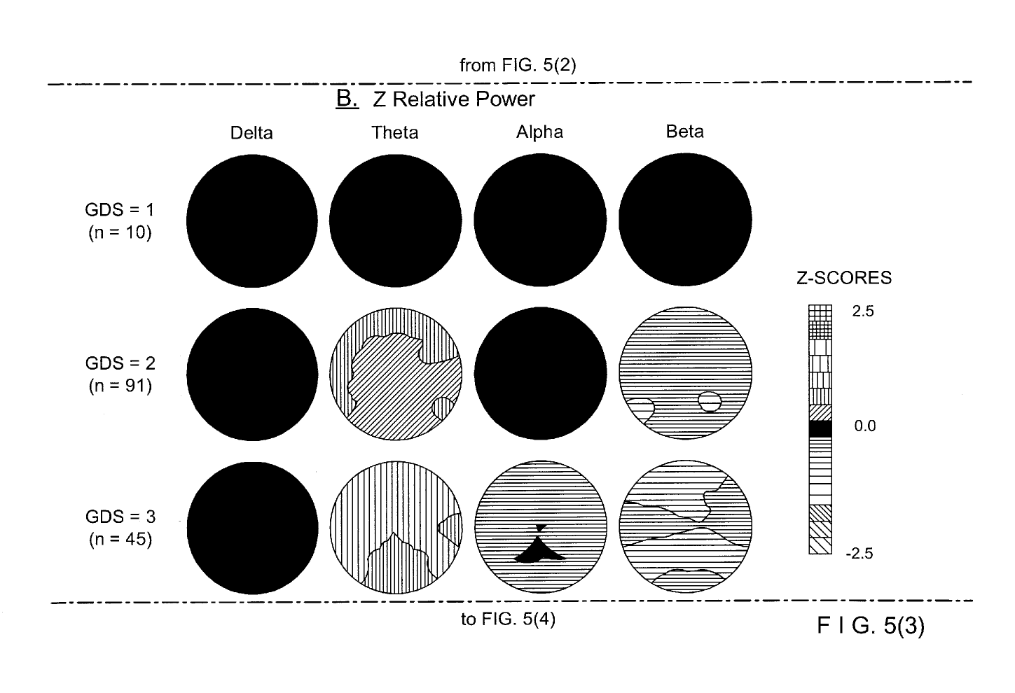

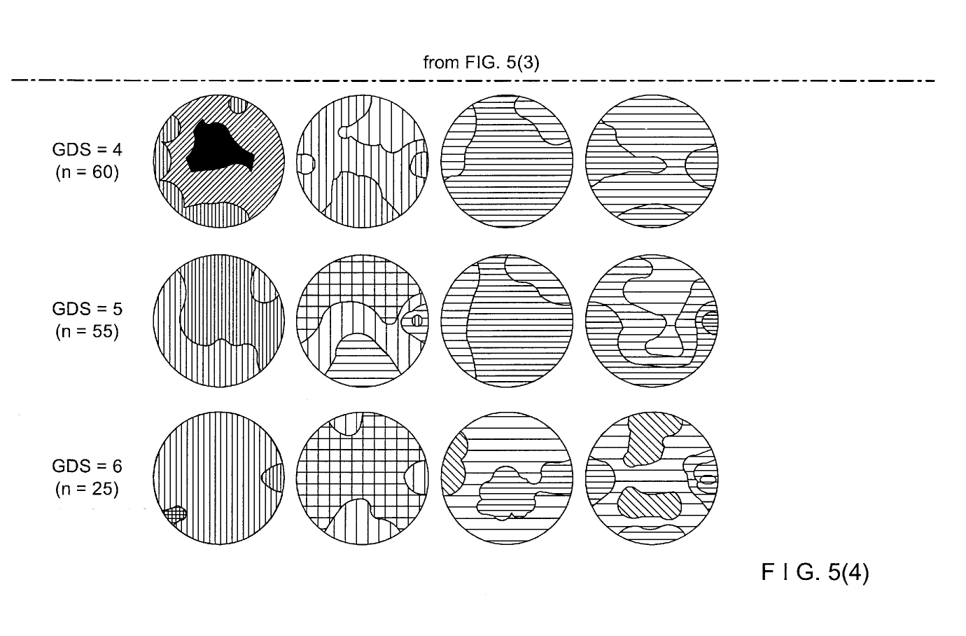

Hippocampal changes in mild cognitive impairment subjects, when assessed using neuroimaging, may predict subsequent AD. Furthermore, changes in tau, the major constituent and pathologic element of the neurofibrillary tangles, as those skilled in the art will understand, increase in MCI subjects in comparison to normal aging subjects at earlier GDS stages, such as GDS stage 2. In addition to the changes between MCI (GDS stage 3) and mild AD (GDS stage 4) and between SCI (GDS stage 2) and MCI (GDS stage 3) subjects, research has demonstrated differences between SCI (GDS stage 2) and similarly aged NCI (GDS stage 1) subjects. For example, research has found electrophysiological differences between GDS stage 1 and GDS stage 2 subjects, as shown in FIG. 5. The continuum of quantitative EEG changes is believed to reflect changes in synaptic and general brain electrophysiological activity including for example, changes in ionotropic receptor activity and general ionic activity. These changes occur as a result of the continuing brain degeneration.

Furthermore, studies in our subjects have recently demonstrated decrements in neurometabolic activity in various brain regions including the parahippocampal gyrus, the middle temporal gyrus, the inferior parietal lobe, the inferior frontal gyrus, the fusiform gyrus, and the thalamus, in SCI subjects (GDS stage 2), in comparison with similarly aged NCI subjects (GDS stage 1) (see Reisberg, et al., The pre-mild cognitive impairment, subjective cognitive impairment stage of Alzheimer's disease. Alzheimer's & Dementia, 2008, 4: (Suppl. 1):S98-S108, for a review). Hence, physiologic differences in SCI versus similarly aged subjects on both electrophysiologic and neurometabolic physiologic parameters have been demonstrated, evidencing the validity of these staging distinctions.

### Establishment of the Time Course of the GDS and FAST Stages in the Brain Aging and Alzheimer's Disease Continuum

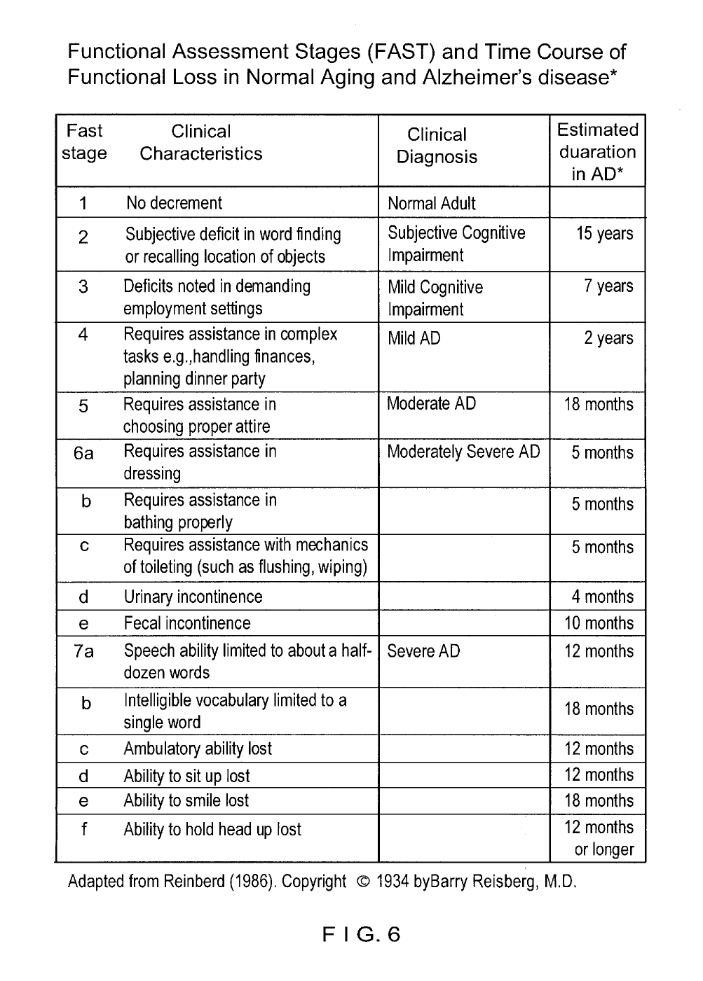

Estimates of the duration of the GDS and FAST stages based upon systematic clinical observations were published in 1986 (see FIG. 6 and Reisberg, Dementia: A subjective approach to identifying reversible causes. Geriatrics, 1986, 41:(4):30-46). The validity of these estimates of the duration of the stages in subjects with “uncomplicated” SCI, MCI, and progressive AD required prospective longitudinal investigations. Specifically, a five year longitudinal study of the course of subjects with probable AD was performed which included subjects with AD residing in the community with a GDS stage of 4 or greater at baseline. A consecutive series of 103 probable AD subjects were studied. Baseline characteristics included a GDS range of stage 4, 5 or 6 (39%, 40% and 21%, of subjects respectively). Follow-ups were then done on 65 locatable and surviving subjects (30 of the subjects were found to be deceased).

A least squares analysis was conducted to estimate the temporal duration of the GDS stages. Since subjects were entered in the GDS 4 stage or greater at baseline, this analysis necessarily underestimated the duration of the GDS 4 stage. Similarly, for GDS stage 7, because of demise and study duration, subjects did not necessarily have time to travel through this stage and, hence, the duration of the GDS 7 stage was necessarily underestimated. The design potentially provided an accurate observation of the duration of the GDS 5 and GDS 6 stages. The observed duration of the GDS stages 5 and 6 using these procedures was 1.4 years and 2.4 years, respectively. These observations were very close to the estimates forwarded a decade earlier (Reisberg et al., “A systematic approach to identifying reversible causes.” Geriatrics, 1986, 41 (4): 30-46). Hence, this longitudinal investigation strongly supported the temporal duration of the GDS 5 and GDS 6 stages from 1986. This least squares analytic procedure found that the GDS 4 stage was longer than 1.6 years and the GDS 7 stage was longer than 1.6 years.

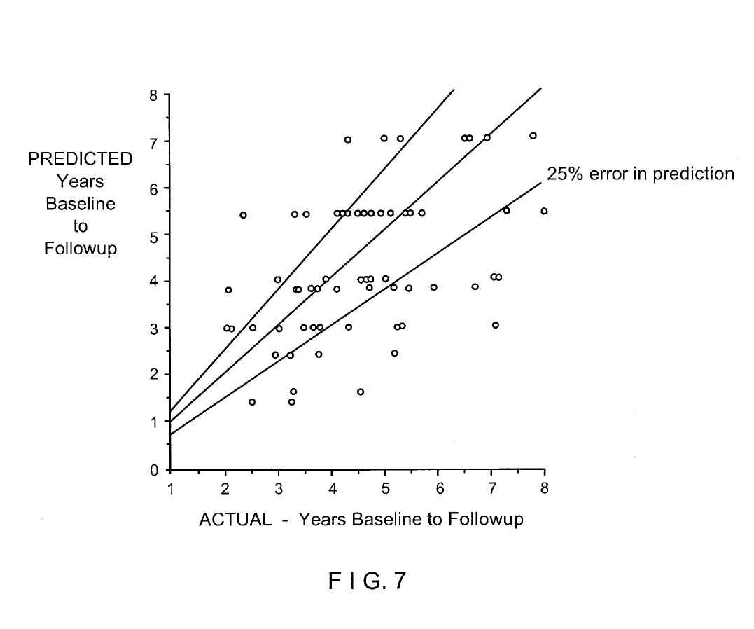

In part because of the limitations in computing the time course of the GDS 4 and GDS 7 stages in this longitudinal study, as described above, other analyses were done which used the time course estimates for the 12 FAST stages and substages, as well as the four GDS stages in scatter plot analyses. Specifically, the relationship between the predicted time that it would take to progress through the respective stages for each of the 65 subjects remaining alive and followed, from baseline to follow-up was computed and combined with the actual observed follow-up times. The predicted temporal durations were the estimated mean stage and substage durations published in the 1986 Geriatrics paper (see FIG. 6). For these calculations the assumption was made, that at baseline and at follow-up, the subjects were at the middle of their respective stage or substage. The results for the GDS stages are shown in FIG. 7. It can be seen that the actual time and the predicted time using the temporal duration estimates were within 25% error limits for a majority of the subjects followed.

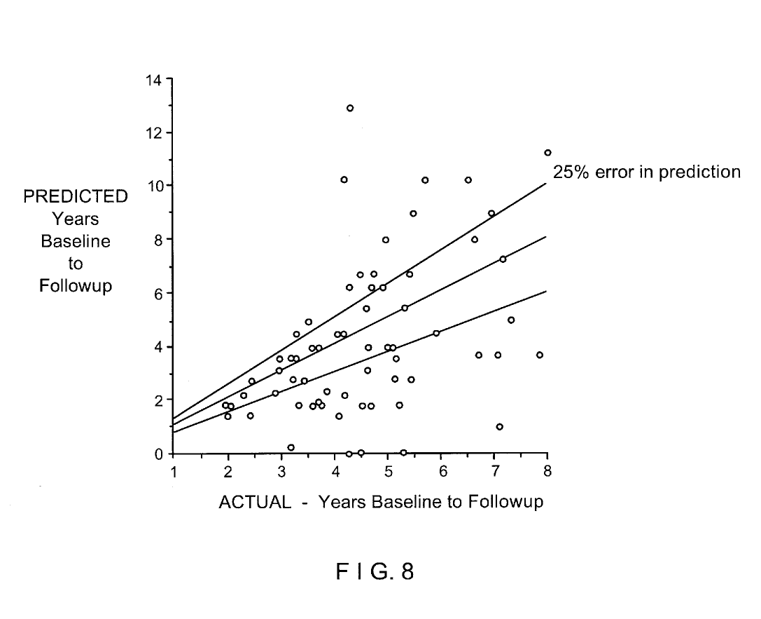

The FAST staging procedures, which are more detailed, and which identify a total of 12 stages and substages in the range of the study, were also used to assess the true duration of the stages of the subjects followed. Since, the GDS and FAST are optimally concordant with the evolution of aging in AD and are also enumerated in an optimally concordant fashion, the FAST temporal estimates are an alternative method for validation of temporal course of AD. For these analyses, the temporal estimates of the FAST substages shown in FIG. 6 were used. In the case of the 4 subjects, who had reached the final 7f FAST substage at follow-up, the assumption was made that survivors had spent a mean of 12 months in this substage. FIG. 8 shows the scatter plot and the variance between the estimated and the actual temporal durations. The mean estimated times from the 1986 predictions and the mean actual times observed were very similar, differing by less than 10%. Hence, this study indicates that the original estimates of the duration of the FAST stages and substages in AD as shown in FIG. 6, appear to be good approximations of the actual mean duration of these stages.

In the case of the MCI stage of the evolution of aging and dementia, the time estimated for the duration of this stage in the 1986 publication was approximately 7 years (Reisberg, B. Dementia: A systematic approach to identifying reversible causes. Geriatrics, 1986, 41 (4): 30-46). Our 1999 longitudinal study of normal aged and otherwise healthy MCI subjects supported the 1986 temporal estimate of duration of the MCI stage. In this study (Kluger, A., Ferris, S. H., Golomb, J., Mittelman, M. S., Reisberg, B. Neuropsychological prediction of decline to dementia in nondemented elderly. Journal of Geriatric Psychiatry and Neurology, 1999, 12:168-179), subjects were followed for a mean of approximately 4 years. Of 87 GDS stage 3 (MCI) subjects at baseline, 67.8% were observed to have declined to dementia at follow-up. Consequently, it was calculated that 17.8% of subjects per year declined from MCI to a general dementia diagnosis. When subjects who did not fulfill the AD diagnostic criteria at follow-up were excluded, it was observed that from a total of 71 GDS stage 3 (MCI) subjects, 66.2% declined to an AD diagnosis. Calculations using the precise follow-up intervals in the study noted a 17.4% per year rate of decline to probable AD. We conclude that the percentages observed for the duration of the MCI stage from this longitudinal study of Kluger et al., are similar to the anticipated rate of decline for an MCI stage with a 7 year mean temporal course. The rate expected if the duration of stage 3 is precisely 7 years would be 14.3% per annum. The small differences observed between the predicted time course and that noted in the Kluger et al., study (i.e., approximately a 3% to 3.5% per year difference in the annual rate of decline) are likely due to subjects presenting somewhat beyond the mean time-point of Stage 3 at the time of evaluation.

Data from the neuropathologic studies also support the approximate seven year temporal duration of the MCI (GDS and FAST stage 3) stage. Since hippocampal regional, volume losses, neuronal cellular losses and the percentage of surviving neurons with neurofibrillary changes all increase linearly with the progression of AD, temporal estimates from these studies which project backward toward normal control values can be compared with actual findings using the 7 year estimate of MCI duration. These studies produce values which are consistent with the suggested 7 year duration of GDS and FAST stage 3 (Bobinski, M., Wegiel, J., Wisniewski, H. M., Tarnawski, M., Reisberg, B., Mlodzik, B., de Leon, M. J., Miller, D. C. Atrophy of hippocampal formation subdivisions correlates with stage and duration of Alzheimer disease. Dementia, 1995, 6:205-210; Bobinski, M., Wegiel, J., Tarnawski, M., Bobinski, M., Reisberg, B., de Leon, M. J., Miller, D. C., Wisniewski, H. M. Relationships between regional neuronal loss and neurofibrillary changes in the hippocampal formation and duration and severity of Alzheimer disease. Journal of Neuropathology and Experimental Neurology, 1997, 56:414-420).

Recent studies are confirming the estimate of the duration of GDS/FAST stage 2 (SCI). Specifically, a recent study evaluated a consecutive series of subjects who had electrophysiologic evaluations and who were followed for a minimum of 7 years who were in GDS stage 2 at baseline (Prichep, L. S., John, E. R., Ferris, S. H., Rausch, L., Fang, Z., Cancro, R., Torossian, C., Reisberg, B. Prediction of longitudinal cognitive decline in normal elderly using electrophysiological imaging. Neurobiology of Aging, 2006, 27: 471-481). The series consisted of 44 subjects. The mean age of these subjects was 72.0 years at baseline (range=64.6-79.8 years). There were 22 females and 22 males in the subject sample. Subjects were assessed to determine whether they declined within the 7 year minimal follow-up time to a GDS stage of 3 or greater. The mean follow-up time of the subjects who were not noted to decline was 8.9±1.8 years (S.D.). For those subjects who did decline, the earliest period at which decline was noted was used as the follow-up time period. Over the 9 year mean observation period, 27 of the 44 subjects were noted to have manifested decline. Hence, this study found that 61.36% of subjects declined over the 8.9 year period. This is a rate of decline of 6.894% per year. If GDS stage 2 (SCI) has a mean duration of 15 years as estimated in our 1986 paper (Reisberg, B. Dementia: A systematic approach to identifying reversible causes. Geriatrics, 1986, 41 (4): 30-46) then we would anticipate a rate of decline of approximately 6.667% per year. Clearly, the observed rate of decline of the GDS stage 2 subjects in the study is very close to the 1986 estimate forwarded 20 years prior to this publication.

### Next Steps the Identification and Advantages of Stage Specific Prognostic Markers

The studies reviewed briefly above, illustrate the robust relationship between the GDS/FAST stages of brain aging and AD and independent “criterion validity” assessments believed to measure the basic processes associated with brain aging with NCI, SCI, MCI and progressive AD. Specifically, these criterion validity elements, reviewed above, include progressive changes in brain electrical activity as measured with electroencephalography (EEG), progressive changes in hippocampal brain volume assessed with both neuroimaging and neuropathologic investigations, progressive changes in neuronal cell counts in the hippocampus assessed neuropathologically, and progressive changes in tau and neurofibrillar pathology assessed through CSF and postmortem neuropathologic studies.

Having established the continuum of pathology in brain aging, SCI, MCI, and progressive AD, the next step relevant to this invention is the identification of the duration of the GDS/FAST stages of these conditions. The system and method of the present invention is directed to the longitudinal study of prognostic outcome in each of the stages of normal aging with NCI, SCI, MCI and mild, moderate, moderately severe and severe AD. These longitudinal studies require observation periods of between approximately 15% and 80% of the stage. These time ranges permit a sufficient and optimal number of subjects to pass through the baseline study range, whereas, there should also be a population of subjects who remain at the baseline stage for an adequate comparison of baseline prognostic markers. Hence, if a baseline population is normally distributed with respect to severity within a particular stage to be studied, then a study lasting 50% of the duration of the stage will produce a follow-up subject population with approximately half of subjects at a subsequent stage at follow-up, and half the subjects remaining at the baseline stage. These two equally numerically robust outcome groups can then be used for the identification of baseline prognostic markers. For example, in SCI, a stage which lasts 15 years, prognostic markers may be identified by prospective observation only after approximately 2 to 12 years have elapsed. Such an observation enables prognostic groups to be identified. In vivo markers, which are stage specific, within each of these prognostic groups may then be used to sensitively identify useful therapies.

An important element of the present discovery is the superiority of the stage specific prognostic markers over cross-sectional changes with the progression of aging and AD pathology. This superiority is based upon the nature of the pathologic mechanism of AD and related degenerative dementias. As already noted, within a particular hippocampal brain region, AD pathology may progress linearly with the temporal progression of the dementia (i.e., over time) as well as with the overall severity of the dementia (i.e., with the progression to subsequent stages). However, the AD pathology is simultaneously spreading to newly involved regions. This is illustrated in a sense in FIG. 5, which shows a pathology in the brain, in terms of incremental slow brain wave activity, spreading to involve progressively more brain regions as subjects go from NCI (stage 1), to SCI (stage 2), to MCI (stage 3), to mild AD (stage 4), to moderate AD (stage 5), to moderately severe AD (stage 6). The brain in essence is progressively, “falling asleep” with increasingly widespread slow wave activity.

This phenomenon of increasingly widespread involvement of brain structure and associated brain physiology is seen from many perspectives. For example, the widely used Braak staging of neuropathology with the evolution of brain aging and dementia is based on progressive spreading of neurofibrillary changes from the entorhinal region of the brain (Braak neuropathologic stages I and II), to more widespread limbic region changes (Braak neuropathologic stages III and IV), to even more widespread neurocortical stages (Braak neuropathologic stages V and VI) (Braak, H., Braak, E., Neuropathological staging of Alzheimer-related changes. Acta Neuropathol., 1991, 82:239-259).

Because of the broadening anatomic progression of AD related pathology with the progression of NCI, to SCI, to MCI, to the stages of AD, cross sectional comparisons of changes are not the most sensitive indicators of, for example, therapeutic change. Rather intrastage prognostic pathologic markers are likely to be much more sensitive indicators of a useful therapy.

An analogy might be useful. The brain of the Alzheimer's patient is “catching fire” neuropathologically. If one wishes to examine the utility of a substance in sensitively putting out the sparks of a fire, one must catch the fire at a specific point in time and space. The extinguishing agent cannot be sensitively studied simultaneously by comparing its ability to put out the initial sparks of a fire and its ability to save the forest.

### Identification of Stage Specific Prognostic Markers

The testing method employed in the current disclosure involved a study of ostensibly normal subjects of a mean age of 72 with subjective cognitive impairments (SCI). The subjects were studied with electroencephalogram (“EEG”) recordings and cognitive evaluations at baseline, which indicated brain electrical activity levels and other criteria essential for the diagnosis of uncomplicated normal aging. Follow-up assessments performed at an interval of at least 7 years were used to categorize the subjects at follow-up as either normal (“NL”, either normal aging with NCI or SCI), MCI or dementia. Standardized low resolution electromagnetic tomographic analyses (“sLORETA”), a quantitative brain imaging method, as those skilled in the art will understand, of the sources of three-dimensional electromagnetic activity detected at the surface were then performed and expressed using images for maximal theta frequency in five subjects randomly chosen from each of these follow-up outcome categories.

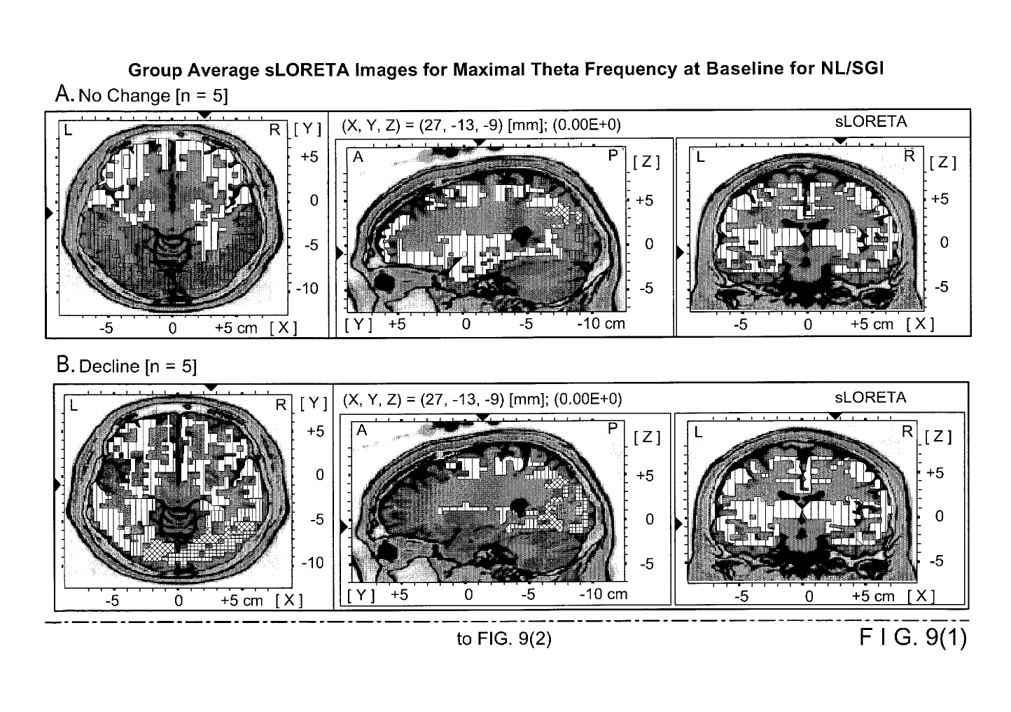

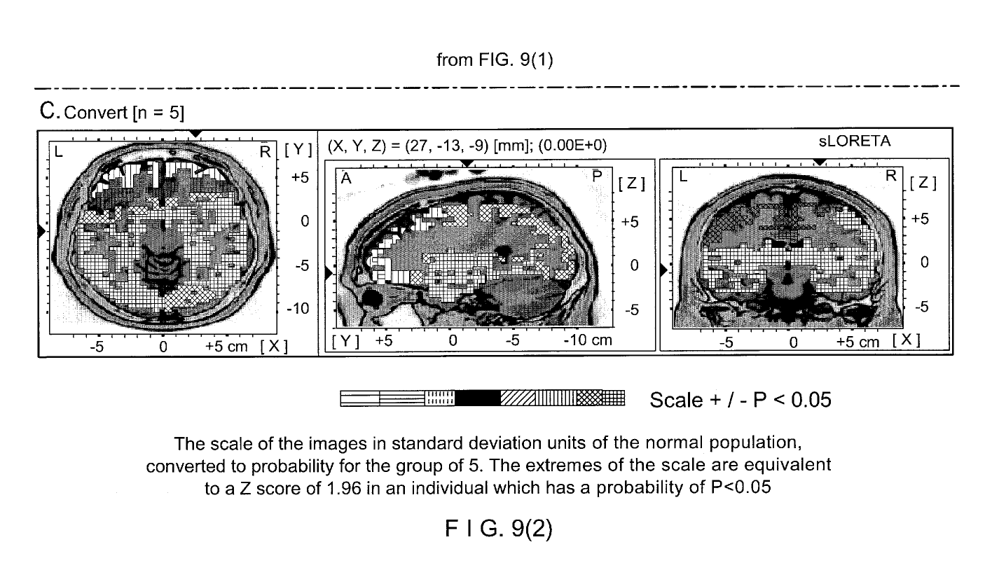

FIG. 9 shows the results of this study. In FIG. 9, voxels are shown with a z scale, which converts p values as indicated in the figure. All voxels are normed relative to age for the age range 16-90 years. Thus, the z scale is relative to age expected normal voxel values for each voxel. In the group average, each individual is normed first and then the group average is constructed. As can be seen from the scale in the figure, the yellow coded voxels indicate maximal theta frequencies differing from normative comparitors. More specifically, in FIG. 9, the scale of the images is in standard deviation units of the normal population, converted to the probability for the group of five. The extremes of the scale are equivalent to a z score of 1.96 in an individual which has a probability of p<0.05.

As shown in FIG. 9, results of the aforementioned study indicated that highly significant differences (p<0.01) in maximal theta frequency were seen at baseline between the three outcome groups. Progressive increments in maximal theta frequency in specific brain regions were seen wherein the maximal theta frequency was highest in subjects who at the time of follow-up were categorized with dementia and lowest in those categorized at the time of follow-up as SCI. Accordingly, the present disclosure noted that decline to MCI and conversion to dementia may be predicted approximately a decade in advance via use of sLORETA procedures. For example, the sLORETA image A indicates no foreseeable change in a subject. sLORETA image B indicates a predicted decline to MCI for a subject. sLORETA image C indicates that a subject may convert to dementia at a later time, wherein a general approximation of ten years may be given. However, it is noted that this time frame may fluctuate to a greater or lesser time. These findings have significant importance for prevention studies and the identification of useful treatment for NCI, SCI, MCI, AD and other degenerative dementias.

### The Method of the Invention

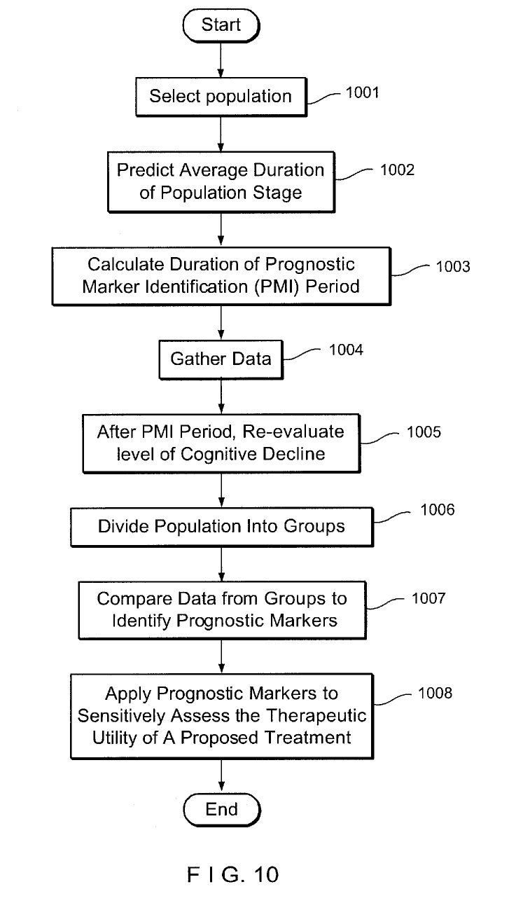

As shown in FIG. 10, a method according to the invention commences in step 1001 with the selection of a population of individuals each of whom is determined to be at a selected stage of cognitive decline (e.g., one of the stages of cognitive decline defined by one of the GDS and FAST systems). In step 1002, a predicted duration of this stage (i.e., a length of time for which the average individual remains at this stage) for the condition and/or disorder for the members of the population is determined in accordance with the method described earlier (e.g., by consulting a look up table). In step 1003, a duration of a prognostic marker identification (“PMI”) period is calculated as a percentage of the stage duration predicted in step 1002 and in step 1004, data is collected from each of the members of the population on a wide range of possible variables. As described above in the application, the PMI period is between approximately 15% and 80% of the predicted duration of the stage for the population. For example, sLORETA data may be collected for each of the members of the population representing a three-dimensional map of electromagnetic activity in the brain (i.e., electromagnetic activity in a series of voxels into which the brain is divided). As would be understood by those skilled in the art, in addition to or as an alternative to the sLORETA data, the data collected may include any or all of a wide range of variables including other forms of electromagnetic data; data regarding brain structure such as grey and white matter distribution or discrimination; data with respect to water diffusivity in brain regions assessed using diffusion tenser imaging or similar procedures, such as diffusion kurtosis imaging; data with respect to the distribution of brain metabolic activity assessed with positron emission tomography (PET scans) or similar methods; data with respect to the assessment of cerebral plaque distribution using the PIB technique; assessment of brain fibrillar staining with FDDNP; chromosomal/genetic information such as data regarding sister chromosome exchanges or mutagenicity in body tissues; chemical analysis of blood, such as for amyloid β (Aβ) levels, or chemical analysis of CSF, such as for Aβ levels or tau levels (total tau and/or tau epitopes); etc. Then in step 1005, after the PMI period has elapsed, the subjects are re-evaluated to determine a degree of cognitive decline since the data was taken and, in step 1006, the population is divided into groups based on the degree of decline over this time. For example, the population may be divided to include a first group of individuals whose level of cognitive decline has remained substantially stable, a second group includes individuals evidencing a low level of decline and a third group evidencing a higher level of decline. Alternatively, a first group may include individuals whose level of cognitive decline has remained substantially stable while a second group includes all individuals who have evidenced additional cognitive decline during the PMI period. Of course, those skilled in the art will understand that any number of groups may be formed. In step 1007, the data from the various groups is then evaluated, for example, using known statistical techniques, to identify prognostic markers which are predictive of decline within the stage. In step 1008, the prognostic markers may then be monitored in patients to make more accurate predictions as to the utility of a proposed treatment intervention.

In addition if desired, subsequent data may be taken for any or all of the variables monitored initially with the exception of those that do not change (e.g., certain forms of genetic information). This subsequent data may then be compared to the initial data to aid in the identification of prognostic markers. Any change in the prognostic marker may be correlated to the time elapsed or to the incremental level of cognitive decline. A therapeutic agent to be evaluated may be administered to a population while monitoring the prognostic markers to determine the efficacy of the agent. For example, if a prognostic marker is represented by a particular level of activity in a selected voxel or group of voxels in a given frequency range, this electromagnetic activity may be monitored before and after administration of the agent to determine if the activity is being adjusted toward the activity level of the group of individuals with slower rates of cognitive decline. Those skilled in the art will understand that, once identified in this manner, the monitoring of these prognostic markers may be utilized for any number of diagnostic or therapeutic processes.

It is contemplated that the system according to the present invention may be used by medical personnel to predict future degenerative dementia in subjects that may or may not currently exhibit symptoms of degenerative dementia without the use of or in addition to neurocognitive tests or subjective clinical evaluations. For example, the system according to the present invention may be used to evaluate elderly ambulatory patients, patients with a family history of degenerative dementia, etc. Furthermore, the present invention may also be useful to identify prognostic markers in subjects with degenerative dementia, or brain aging related conditions whose cognitive impairment is not associated, or not entirely associated with AD pathology. For example, the system according to the present invention may be used to evaluate patients having cerebrovascular dementia or other brain conditions known to cause degenerative dementias such as frontotemporal dementias (including Pick's disease, corticobasilar degeneration, progressive aphasia, semantic dementia, FTDP-17, frontotemporal dementia with motor neuron disease, and frontotemporal dementia without specific pathology), Lewy body dementia, Parkinsonian dementia, and dementia associated with presenillin or APP mutations.

As described above, having determined the duration of the stages, the next step is to establish prognostic subgroups in a stage specific manner for putative markers. To establish these prognostic subgroups, subjects are followed for a time period adequate for subgroup identification. An adequate observation period is approximately 15 to 80% of the predicted duration of the stage or substage. Therefore, to establish prognostic subgroups, the minimum observation period for GDS stage 2 subjects is approximately 2.25 years. The optimal observation period for prognostic subgroup identification is approximately 4-10 years. The 1986 estimates of the duration of the respective stages and substages (see FIG. 6) can be used together with this formula, to establish the longitudinal observation time necessary for the identification of prognostic markers.

Various in vivo markers which proceed in a progressive manner with the evolution of dementia pathology can be applied using the methodology described above. Examples of such markers are neurometabolic assessments of brain change, neuroanatomic assessments of brain change, electrophysiologic assessments of brain change, electromagnetic assessments of brain change, magnetic assessments of brain change, and measures of progressive dementia related pathology such as progressive neurofibrillar and more general fibrillar brain changes, changes in cerebral and peripheral proteins such as beta amyloid, as well as other proteins, changes in cerebral grey to white matter ratios, as well as peripheral markers of progressive dementia related pathology such as proteomic changes in various tissues, such as lymphocytes and other blood cells and blood serum, genetic and chromosomal changes in peripheral tissues, changes in protein and/or RNA expression in various tissues, cerebrospinal fluid markers of progressive AD pathology such as changes in beta-amyloid and tau as well as specific APP and tau derivatives and tau kinases and epitopes, various blood markers of progressive pathology including beta-amyloid and tau, and other markers of progressive pathology in AD. These procedures can also be applied to measures reflecting progressive decrements in cerebral processing and performance in patients with the brain aging to dementia continuum. An example of a change in cerebral performance would be motoric, balance, co-ordination, speed of processing, and performance based tests, and other psychometric and motor and behavioral performance measures.

Investigative tools which are presently used for marker identification include positron emission tomography (PET) scans, PET performed after injection of 2-(1-{6-[(2-[F-18]fluoroethyl)(methyl)amino]-2-naphthyl}ethylidene) malononitrile (FDDNP), and an amyloid-imaging, PET tracer, termed Pittsburgh Compound-B (PIB), magnetic resonance imaging (MRI), diffusion tensor imaging (DTI), ), diffusion kurtosis imaging (DKI), functional magnetic resonance imaging (fMRI), single photon emission tomography (SPECT), quantitative analysis of electroencephalographic rhythms (QEEG), the presence of abnormal cerebral activity (such as spikes, sharp waves, bursts, etc.), some of which may be characteristic of epileptiform activity, electrical evoked potential activity (EP) and averaged evoked potentials (AEP), regional cerebral blood flow and metabolism (rCBF), low resolution brain electromagnetic tomography (LORETA), variable resolution electromagnetic tomography (VARETA), magnetoencephalogram (MEG) (sometimes called MSI—magnetic source imaging) measures of brain electrical activity including those using superconductive quantum interference devices (SQUID), simple and disjunctive computerized head tracking devices, assessments of the extent of mutagenicity and the magnitude of sister chromatid exchanges in lymphocytes, amongst others.

The progressive markers, such as those mentioned above, are then employed in a prognostic study in subjects with brain aging, NCI, SCI, MCI, and/or a stage or substage of AD, or, more generally, progressive dementia, for the period of time recommended depending upon the stage or substage to be examined.

Stage specific outcome groups are then identified. These are identified based upon whether the subjects in the group remained at the same stage over the duration of the follow-up period, or whether the subjects progressed to a more advanced stage. In this portion of the invention, one option is the identification of two (dichotomous) outcome groups, i.e., (1) subjects who remained the same and (2) subjects who declined by one stage or more. Another option is the identification of three outcome groups, i.e., (1) subjects who remained the same, and (2) subjects who declined by one stage, and (3) subjects who declined by two stages or more. Other, more complex outcomes based on stage specific stability or decline, are also possible.

The next step is that, having identified the outcome groups, one then goes back to the original baseline data and examines the putative prognostic marker in the subsequently determined outcome groups. Markers which distinguish the outcome groups at baseline, within the initial baseline single stage, are identified.

The baseline prognostic markers can be identified using qualitative procedures or quantitative procedures. For example, in the case of a PET scan, one can examine a putative prognostic marker of regional metabolic activity based upon color coding of metabolic activity in three dimensional brain images. Ideally, these images are modeled as regions of interest (ROIs) or as volume elements (voxels). A pre-identified ROI or voxel or collection of ROIs or voxels is compared and contrasted in corresponding brain geographic regions in the pre-identified prognostic outcome groups. In the case of PET, the difference in brain metabolic activity at baseline in the subjects with the diverse outcomes for the particular voxel is the prognostic marker to be used subsequently in the therapeutic trial (PMTT).

Having identified the PMTT, this marker and other identified PMTTs are utilized at baseline in a therapeutic trial. The trial may be of a pharmacotherapeutic agent or a nonpharmacotherapeutic agent. For example, the trial may examine the efficacy of exercise in preventing the progression of SCI to MCI. Pre-selected PMTTs are employed, depending upon the stage to be examined. As a result of the procedures described above, the sensitivity of the PMTT markers is greatly enhanced in comparison with currently available procedures.

Current procedures invariably employ severity markers rather than PMTT markers. The difference is that the severity markers are selected on the basis of cross-sectional severity studies. They are not selected on the basis of baseline, within stage, prognostic marker studies. The latter PMTTs take much more time and effort to develop. Once developed, however, the PMTTs are much more sensitive to the process of brain aging and dementia than the severity markers. The reason for this is that the process of brain aging and progressive dementia frequently proceeds linearly within a given brain structure such as a hippocampal brain region, as described clearly in our work (Bobinski, M., Wegiel, J., Wisniewski, H. M., Tarnawski, M., Reisberg, B., Mlodzik, B., de Leon, M. J., Miller, D. C. Atrophy of hippocampal formation subdivisions correlates with stage and duration of Alzheimer disease. Dementia, 1995, 6:205-210; Bobinski, M., Wegiel, J., Tarnawski, M., Bobinski, M., Reisberg, B., de Leon, M. J., Miller, D. C., Wisniewski, H. M. Relationships between regional neuronal loss and neurofibrillary changes in the hippocampal formation and duration and severity of Alzheimer disease. Journal of Neuropathology and Experimental Neurology, 1997, 56:414-420). In addition to this linear progression, the process also spreads to other brain structures including adjacent and distal structures. Hence, cross-sectional comparisons between stages are comparatively less sensitive than the prognostic markers obtained using the methods described in this discovery.

To identify the PMTTs, one can also use brain atlases to better identify specific regions in the sLORETA images. For example, the classical sLORETA procedures use thousands of dipole sources within a 3-D brain model which is coregistered into Talairach space (Talairach and Tournoux, 1988. Co-Plannar Stereotaxic Altas of the Human Brain. Thieme, Stuttgart). The cortex is then modeled as a collection of voxels in the digitized Talairach atlas. These LORETA procedures find the linear inverse solutions that maximize the synchronization of strength between neighboring neuronal populations. This roughly corresponds to the 3-D distribution of neuronal activity that has maximum synchronization in terms of orientation and strength among neighboring neuronal populations. Hence, in this invention markers are identified depending upon the differences between group 1 (no change) and group 2 (decline to MCI) and group 3 (conversion to dementia). The markers consist of voxels at baseline in the LORETA/Talairach space. A putative therapeutic agent is subsequently studied at this stage using the PMTTs. A therapeutic agent would mitigate the group 1 to group 2 and/or group 2 to group 3, and/or group 1 to group 3 differential (see FIG. 9) of the PMTT. In particular situations, adjacent stage PMTT or combinations of PMTTs might also be useful. As a result of these procedures, the discovery of useful therapeutic agents for the brain aging and AD continuum is greatly sensitized and enhanced in terms of ease of identification of therapies.

While specific embodiments of the invention have been illustrated and described herein, it is realized that numerous modifications and changes will occur to those skilled in the art. Those skilled in the art will recognize that benefits may also arise from the use of prognostic markers found in regard to one stage with respect to contiguous stages. For example, prognostic markers found relevant to decline during stage 2 may be useful in conjunction with prognostic markers for stage 3 in examining the transition period between these stages for therapeutic purposes. It is therefore to be understood that the appended claims are intended to cover all such modifications and changes as fall within the true spirit and scope of the invention.

What is claimed is:

1. A method for identifying markers sensitive to cognitive decline, comprising the steps of:
predicting for a population of first individuals at a common stage of cognitive decline a stage length as an average length of time during which the first individuals will remain in the current stage of cognitive decline;
calculating a duration of a prognostic marker identification period based on the stage length;
gathering from the population data relevant to a plurality of potential prognostic markers;
evaluating the first individuals after the stage length has elapsed to determine for each individual a degree of cognitive decline during the stage length;
dividing the population into a plurality of groups, each group corresponding to a degree of cognitive decline during the stage length; and
comparing the data from the groups to identify prognostic markers from the plurality of potential prognostic markers.

2. The method of claim 1, wherein the data gathered from the population includes one of genetic changes, chromosomal changes, data corresponding to brain electrical activity and data corresponding to brain metabolic activity.

3. The method of claim 1, wherein the data gathered from the population corresponds to one of neuroanatomic changes, changes in brain water distribution and diffusivity, regional cerebral blood flow, brain electromagnetic activity and brain magnetic activity.

4. The method of claim 1, wherein the data gathered from the population corresponds to one of progressive neurofibrillary changes, general brain fibrillar changes, proteomic changes, changes in cerebral gray to white matter ratios, changes in RNA expression, changes in amyloid, changes in beta-amyloid, changes in amyloid precursor protein, changes in tau.

5. The method of claim 2, wherein gathering data includes one of positron emission tomography (PET) scanning, magnetic resonance imaging (MRI), functional magnetic resonance imaging (fMRI), diffusion tensor imaging (DTI), diffusion kurtosis imaging (DKI), single photon emission tomography (SPECT), analysis of regional cerebral blood flow and metabolism (rCBF), magnetoencephalography (MEG).

6. The method of claim 2, wherein gathering data includes one of an amyloid-imaging, PET tracer, terminal Pittsburgh Compound-B (PIB) imaging,

7. The method of claim 6, wherein gathering data includes a PET tracer termed 2-(1-{6-[(2-[F-18]fluoroethyl)(methyl)amino]-2-naphthyl} ethylidene) malononitrile (FDDNP).

8. The method of claim 2, wherein gathering data includes one of quantitative analysis of electroencephalographic rhythms (qEEG), detection of abnormal cerebral activity, analysis of electrical evoked potentials, low resolution brain electromagnetic tomography (LORETA), variable resolution electromagnetic tomography (VARETA), superconductive quantam interference (SQUID).

9. The method of claim 1, wherein the data is analyzed to correspond to brain activity in each of a plurality of voxels of brains of at least a portion of the first individuals.

10. The method of claim 1, wherein the data is analyzed to correspond to electrical activity between adjacent voxels of brains of at least a portion of the first individuals.

11. The method of claim 1, wherein data is analyzed to correspond to brain activity in each of a plurality of regions of interest in brains of at least a portion of the first individuals.

12. The method of claim 9, wherein the portion of first individuals includes individuals from first and second ones of the groups.

13. The method of claim 11, wherein the portion of first individuals includes individuals for first and second ones of the groups.

14. The method of claim 1, wherein the duration of the prognostic marker identification period is calculated as a range of times as a percentage of the stage length.

15. The method of claim 14, wherein the prognostic marker identification period is between 15% and 80% of the stage length.

16. The method of claim 1, further comprising administering to a first one of the groups exhibiting cognitive decline during the stage length increased with respect to a second one of the groups a therapeutic agent targeted to an identified prognostic marker.

17. The method of claim 1, further comprising administering to a test group a therapeutic agent and evaluating a change in an identified prognostic marker over a period of time to determine an efficacy of the therapeutic agent in reducing a rate of cognitive decline.

18. The method of claim 1, further comprising gathering for the individuals data relevant to additional factors relevant to cognitive decline and adjusting the evaluated degree of cognitive decline for the individuals to account for the additional factors prior to dividing the individuals into groups.

19. The method of claim 18, wherein the additional factors include at least one of age of the individuals, genetic markers relevant to rates of cognitive decline.

20. The method of claim 2, wherein gathering data includes one of cellular mutagenicity, cellular sister chromosome exchange, cerebral electrical spike activity, cerebral sharp wave activity, cerebral burst activity.

21. A method of identifying therapeutic compounds to treat cognitive decline, comprising the steps of:
predicting for a first population of individuals at a common stage of cognitive decline a stage length as an average length of time during which the first individuals will remain in the current stage of cognitive decline;
calculating a duration of a prognostic marker identification period based on the stage length;
gathering from the population data relevant to a plurality of potential prognostic markers;
evaluating the individuals after the stage length has elapsed to determine for each first individual a degree of cognitive decline during the stage length;
dividing the first population into a plurality of groups, each group corresponding to a degree of cognitive decline during the stage length;
comparing the data from the groups to identify prognostic markers from the plurality of potential prognostic markers;
applying a therapeutic agent to a second population of individuals; and
applying to a second population of individuals a therapeutic agent targeted to a first one of the identified prognostic markers.
